(19) United States
(12) Patent Application Publication
(10) Pub. No.: US 2013/0345325 A1
(43) Pub. Date: Dec. 26, 2013

(54) METHOD FOR PRODUCING METHANOL OR HYDROCARBONS FROM A CARBON MATERIAL, INCLUDING A REFORMING STEP, THE OPERATING CONDITIONS OF WHICH ARE SELECTIVELY ADJUSTED

(71) Applicants: Michel Lecomte, Rueil Malmaison (FR); Alejandro Carlos Mourgues Codern, Le Creusot (FR)

(72) Inventors: Michel Lecomte, Rueil Malmaison (FR); Alejandro Carlos Mourgues Codern, Le Creusot (FR)

(73) Assignee: AREVA, Paris (FR)

(21) Appl. No.: 14/000,668

(22) PCT Filed: Feb. 22, 2012
(86) PCT No.: PCT/EP12/53014

§ 371 Date: Sep. 12, 2013

(30) Foreign Application Priority Data
Feb. 22, 2011 (FR) 1151408

(51) Int. Cl. C07C 29/151; C07C 1/04

(52) U.S. Cl. CPC C07C 29/1518; C07C 1/0455
USPC 518/702; 422/162

(57) ABSTRACT

A method for producing methanol or hydrocarbons is provided. The production method comprises producing a synthesis gas from carbonaceous material, according to a method comprising at least one reforming step, the synthesis gas having a first hydrogen/carbon monoxide molar ratio under the first operating conditions for the reforming operation; producing a stream of hydrogen from a hydrogenated raw material and from a first consumed electric power, the hydrogen stream having a first molar flow rate for said first consumed electric power; and lowering the consumed electric power for producing the hydrogen stream, down to a second electric power below the first electric power and transitioning to second operating conditions different from the first for the reforming operation in order to compensate for the lowering of the molar flow rate of the hydrogen flow, the synthesis gas having a second hydrogen/carbon monoxide molar ratio greater than the first under the second operating conditions.

According to a first aspect, the invention relates to a method for producing methanol or hydrocarbons from at least one carbonaceous material.

### BACKGROUND

US 2010/0022669 describes a method for producing liquid hydrocarbons from a carbonaceous material such as methane or biomass, by using a source of renewable energy of variable power, a unit for generating oxygen (air separation unit or electrolyzer) consuming a fraction of the electric energy, a reformer consuming the generated oxygen and another fraction of the electric energy, and a unit for making liquid hydrocarbons from synthetic gas produced by the reformer.

This document envisions several solutions for solving the problems of the variability of the available electric power. A first envisioned solution is to vary the amount of oxygen provided to the reformer, in order to make the latter more or less endothermic or exothermic and modify the electric power required for the heating. Other solutions consist of lowering the production capacity of liquid hydrocarbons, using an additional electric source for heating the reformer, using an additional heat source or further using an oxygen buffer storage.

These methods are not very efficient, and make it difficult to compensate for a significant decrease in the available long term electric power.

### SUMMARY OF THE INVENTION

In this context, the invention aimed at proposing a method which is more robust, and which may tolerate longer and more pronounced decreases in electric power.

For this purpose, the invention relates to a method for producing methanol or hydrocarbons from at least one carbonaceous material, the method comprising the following steps:
producing a synthesis gas stream from the carbonaceous material according to a method comprising at least one operation for reforming an intermediate gas stream stemming from the carbonaceous material, the synthesis gas stream comprising at least hydrogen and carbon monoxide, the synthesis gas having a first hydrogen/carbon monoxide molar ratio under first operating conditions for the reforming operation;
producing a hydrogen stream from a hydrogenated raw material and from a first consumed electric power, the hydrogen stream having a first molar flow rate for said first consumed electric power;
producing methanol or hydrocarbons from the synthesis gas stream and from the hydrogen stream;
lowering the consumed electric power in order to produce the hydrogen stream, down to a second electric power lower than the first electric power, the hydrogen stream having a second molar flow rate below the first molar flow rate for the second consumed electric power, and being set under second operating conditions different from the first for the reforming operation in order to compensate for the decrease in the molar flow rate of the hydrogen stream, the synthesis gas having a second hydrogen/carbon monoxide molar ratio which is greater than the first under the second operating conditions.

The method may further include one or several of the characteristics below, considered individually or according to all technically possible combinations:
under the first operating conditions, the reforming operation is carried out at a first temperature; under the second operating conditions, the reforming operation is carried out at a second temperature below the first temperature;
under the first operating conditions, the reforming operation is carried out at a first pressure; under the second operating conditions, the reforming operation is carried out at a second pressure below the first pressure;
the reforming operation is carried out in a reforming unit receiving as an input a plurality of inflows containing organic molecules, including the intermediate stream, the reforming unit receiving from the different inflows, all in all, a total molar flow of carbon atoms contained in the organic molecules and a total water H₂O molar flow, the reforming operation passing from the first operating conditions to the second operating conditions by modification of a ratio of the total water H₂O molar flow rate over the total molar flow rate of carbon atoms contained in the organic molecules;
the ratio of the total water H₂O molar flow rate over the total molar flow rate of carbon atoms contained in the organic molecules is equal to a first value under the first operating conditions, and is equal to a second value greater than the first under the second operating conditions;
the second value is comprised between 1.1 and 5 times the first value, preferably between 1.5 and 3 times the first value;
the reforming operation is carried out in a reforming unit receiving as an input a plurality of inflows, including the intermediate stream, the reforming unit receiving different inflows, all in all, a total molar flow rate of carbon atoms contained in the organic molecules and a total oxygen O₂ molar flow rate, the reforming operation passing from the first operating conditions to the second operating conditions by modification of a ratio of the total molar flow rate of oxygen over the total flow rate of carbon atoms contained in the organic molecules;
the hydrogen stream is produced from electricity provided by an electric distribution network serving other consumers; and
the intermediate stream is obtained by gasification of the carbonaceous material.

According to a second aspect, the invention deals with a facility for producing methanol or hydrocarbons from at least one carbonaceous material, the facility comprising:
a unit for producing a synthesis gas stream from the carbonaceous material, comprising at least one unit for reforming an intermediate gas stream stemming from the carbonaceous material, the synthetic gas stream comprising at least hydrogen and carbon monoxide, the synthesis gas having a first hydrogen/carbon monoxide molar ratio under the first operating conditions for the reforming operation;
a unit for producing a hydrogen stream from a hydrogenated raw material and from a first consumed electric power, the hydrogen stream having a first molar flow rate for said consumed electric power;
a unit for producing methanol or hydrocarbons from the synthetic gas stream and from the hydrogen stream;
a programmed control unit for decreasing the consumed electric power in order to produce the hydrogen stream, down to a second electric power below the first electric power, the hydrogen stream having a second molar flow rate below the first molar flow rate for the second consumed electric power, the control unit being programmed so as to have the reforming unit pass under second operating conditions different from the first for compensating for the decrease in the molar ratio of the hydrogen stream, the synthesis gas having a second hydrogen/carbon monoxide molar ratio greater than the first under the second operating conditions.

### BRIEF SUMMARY OF THE DRAWINGS

Other features and advantages of the invention will become apparent from the detailed description which is given thereof below, as an indication and by no means as a limitation, with reference to the appended figures, wherein:

FIG. 1 is a simplified schematic illustration of a facility for producing hydrocarbons according to a first embodiment of the invention;

FIG. 2 is a schematic illustration similar to the one of FIG. 1, for a production facility according to a second embodiment of the invention;

FIGS. 3 to 8 are graphic illustrations of the time-dependent change in the molar fractions on a dry basis of CH₄, H₂, CO and CO₂ at equilibrium and of the conversion rate of methane at the outlet of the reformer, versus the reaction temperature, at 30 bars, for different input conditions in the reformer;

FIGS. 9 and 10 are graphs illustrating the time-dependent change of the CO/CO₂ and H₂/Co ratios at equilibrium, versus the reaction temperature, at 30 bars for a reformer operating under the operating conditions of FIGS. 3 to 8.

### DETAILED DESCRIPTION

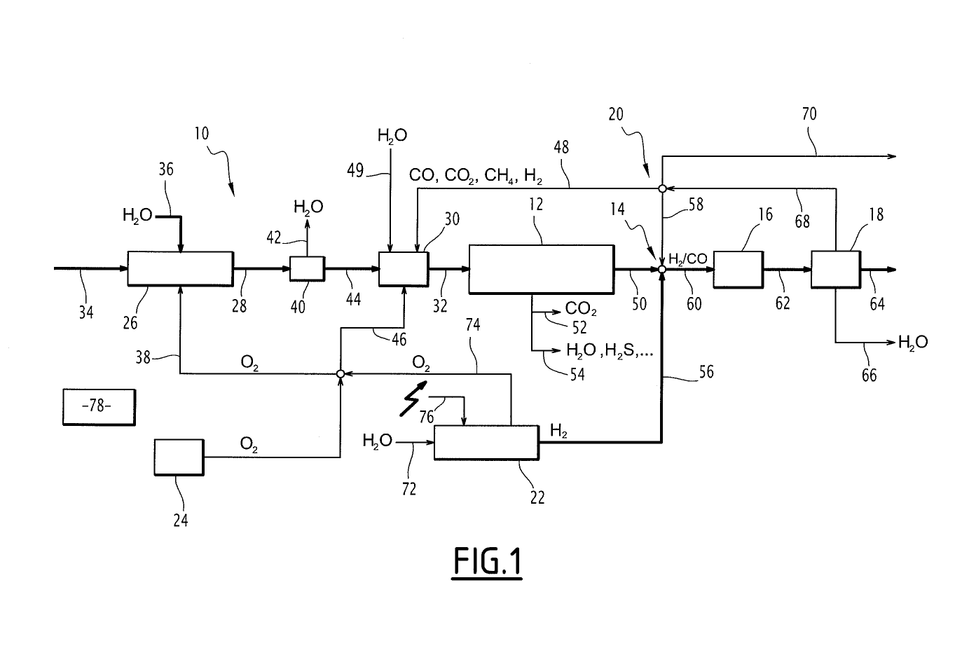

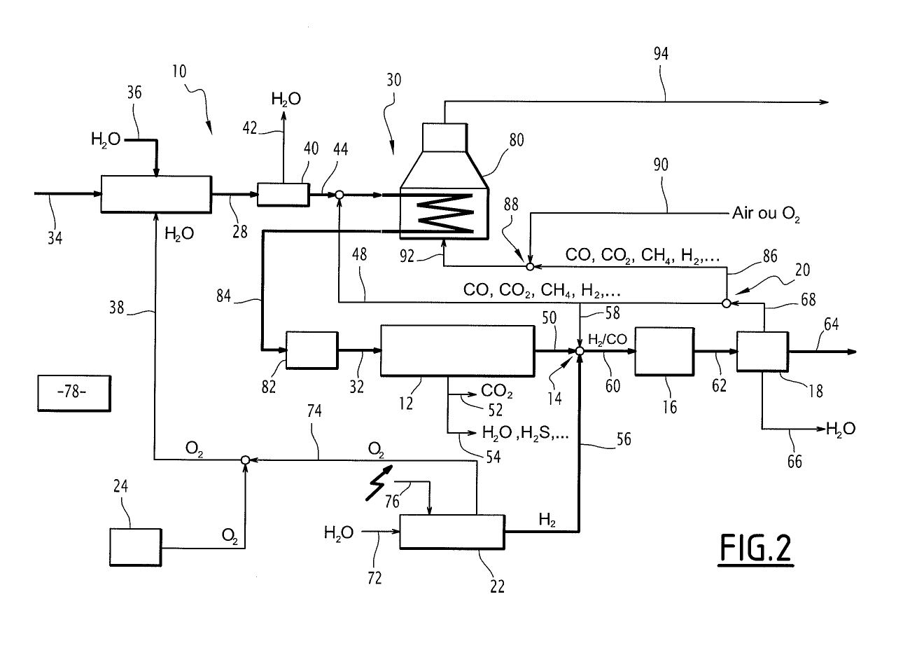

In FIGS. 1 and 2, each rectangle corresponds both to a step or a sub-step of the method of the invention, and to the corresponding industrial facility unit. The facility will be described as comprising units corresponding to the steps or sub-steps of the method.

The facilities schematically illustrated in FIGS. 1 and 2 aim at producing methanol and/or hydrocarbons from a carbonaceous material. The carbonaceous material may comprise one or more of the following elements:
coal, lignite,
municipal waste,
animal waste,
biomass,
plastic materials such as polyethylene or polyethylene terephthalate etc.

This list is non-limiting.

In the exemplary embodiment of FIG. 1, the carbonaceous material is biomass.

The facility of FIG. 1 comprises at least the following units:
a unit 10 for producing a first stream of synthetic gas from the carbonaceous material,
a unit 12 for conditioning the first stream of synthetic gas;
a unit 14 for adding a hydrogen stream to the first stream of synthesis gas, in order to form a second stream of synthesis gas;
a unit 16 for producing a first product stream from the second stream of synthesis gas;
optionally a first separation unit 18, in which the first product stream is separated, for example into a water stream, into a second product stream and into a first stream of gas intended to be recycled;
optionally a second separation unit 20, in which the first gas stream intended to be recycled is separated into a gas purge stream and into first and second recycled streams;
a unit 22 for electrolysis of water;
optionally an air separation unit 24.

The unit 10 for producing the first stream of synthesis gas typically comprises a unit 26 for gasification of the biomass producing an intermediate flow, and a reforming unit 30 producing the first stream of synthesis gas from the intermediate flow.

The biomass may optionally undergo pretreatment before feeding the gasification unit 26. This pretreatment may be a milling, drying operation or any other operation required for putting the biomass in a suitable condition so as to be treated in the gasification unit.

The gasification unit is selected according to the characteristics of the biomass to be treated. It is of known type, it will not be described in more detail here. It is fed with biomass via line 34, with water via line 36 and with oxygen via line 38. The oxygen stems from the electrolysis unit 22 and/or from the air separation unit 24. The intermediate gas stream leaves the gasification unit through line 28. In the gasification unit 26, the biomass is broken down into a gas essentially comprising methane CH₄, water, carbon dioxide, carbon monoxide and hydrogen.

The intermediate stream then passes into a unit 40 for adjusting the steam level in the intermediate stream. If the intermediate stream contains an excess of steam relatively to the level required for proper operation of the reforming unit 30, a portion of the steam is separated from the intermediate stream and leaves the unit 40 through line 42. The intermediate stream leaves the unit 40 through the line 44, and feeds the reforming unit 30. The water stream leaving the unit 40 through the line 42 may feed the gasification unit 26 or the electrolysis unit 22.

The reforming unit is typically an autothermal reforming (ATR) unit or a partial oxidation reforming (PDX: Partial Oxidation) unit or a steam reforming (SR) unit. The reforming unit 30 is supplied with the intermediate stream via line 44, and is supplied with oxygen from the electrolysis unit 22 or from the air separation unit 24, via line 46. It also receives the first recycled gas stream via line 48. It is optionally supplied with steam through line 49, in the case when the intermediate stream does not comprise sufficient steam for properly operating the reforming unit 30. The first recycled gas stream essentially comprises CO, CO₂, CH₄, H₂ and light hydrocarbons. In the reforming unit 30, the methane molecules from the intermediate stream and the methane molecules as well as the light hydrocarbons stemming from the first recycled gas stream are broken down and converted into CO, CO₂ and H₂.

The reforming unit produces the first stream of synthesis gas, which is directed towards the unit for conditioning the gases 12 via line 32.

The first stream of synthesis gas in majority comprises carbon monoxide CO, carbon dioxide CO₂, hydrogen H₂ and water. It also comprises other gases in a smaller amount. These other gases are i.a. non-converted hydrocarbons and impurities (H₂S, NOx, etc).

The unit for conditioning the gases 12 receives the first stream of synthesis gas stemming from the reforming unit 30 and separates the carbon monoxide and the hydrogen from the carbon dioxide, steam and other impurities such as H₂S for example. In the unit for conditioning the gases 12, the first stream of synthesis gas is thus separated into a first stream of purified synthesis gas essentially containing CO and hydrogen, and into one or several separate gas streams. The first stream of purified gas leaves the unit 12 via line 50, the separate gas streams via lines 52 and 54.

The first stream of purified synthesis gas is directed from the gas conditioning unit 12 as far as the unit 14 for adding hydrogen, via line 50. The separate gas streams, before being discarded into the atmosphere, may undergo a treatment for example aiming at recovering the steam so as to feed the electrolysis unit 22 or the gasification unit 26. The CO₂ may be discharged into the atmosphere or recycled or stored.

In the unit 14, a determined amount of hydrogen is added to the first stream of purified synthesis gas so as to form a second stream of synthesis gas. The hydrogen stems from the electrolysis unit 22 via line 56. In the unit 14, the second stream of recycled gas is also mixed with hydrogen and with the first stream of purified synthesis gas. The second stream of recycled gas feeds the unit 14 via line 58. This second stream mainly comprises H₂ and CO, and smaller amounts of CO₂, methane and light hydrocarbons. The second stream of synthesis gas leaves the unit 14 via line 60 and feeds the production unit 16.

The production unit 16, in the exemplary embodiment of FIG. 1, operates according to the Fisher-Tropsch process. In this unit, the carbon dioxide CO reacts with hydrogen for producing hydrocarbons. This reaction is catalyzed with suitable catalysts. The production unit is of a known type and will not be described in more details here.

The first product stream leaves the unit 16 via line 62 and is directed as far as the first separation unit 18. The first product stream comprises the CO and H₂ fraction stemming from the second stream of synthesis gas and not having reacted in the unit 16. It also comprises all the products stemming from reactions within the unit 16, and in particular a large number of hydrocarbons of different natures, as well as water. It further comprises i.a. methane CH₄, and C₂, C₃, C₄, . . . , C₁₀₀, chains etc. The hydrocarbons comprise i.a. naphthas, ceraceous materials, waxes (ceraceous materials) and recyclable products such as for example diesel fuel, and/or kerosene, etc.

In the first separation unit, the first product stream is separated into three streams:
a water stream with certain organic products dissolved in a small proportion (alcohols, organic acids);
a second stream of final products comprising the major part of the condensable products (for the Fischer-Tropsch process, C₄ to C₁₀₀ products);
a first gas stream intended to be recycled, comprising at least CO, CO₂, H₂ and light hydrocarbons, notably CH₄.

The second stream of final product leaves the first separation unit 18 via line 64. It may be directed towards a post-treatment unit aiming at separating the different components of this stream from each other. The post-treatment unit may also be a unit aiming at converting these components into another recyclable product, for example diesel fuel, etc.

The water stream leaves the first separation unit 18 via line 66. For example it is directed towards the electrolysis unit 22 or towards the gasification unit 26, optionally after treatment. The first stream of gas intended to be recycled leaves the unit 18 through line 68 and is directed towards the second separation unit 20.

In this second separation unit, this stream is divided into three different streams: the purge stream (line 70), the recycling stream towards the unit 14 via line 58 and the recycling stream to the unit 30 via line 48. The flow rate of each stream is selected so as to optimize the yield of the process. Normally, the value of the purge flow rate is the smallest of the three. In an exemplary embodiment, the three flows have exactly the same composition for all the compounds. However, a separation unit may be provided with which it is possible to efficiently remove the impurities from the recycling streams towards the units 14 and 30, and to transfer them into the purge stream.

The purge stream is discharged into the atmosphere, after optional post-treatment. This post-treatment may consist of burning the light hydrocarbons (energy recycling), separating certain gas elements for which emission into the atmosphere is regulated etc.

The electrolysis unit 22 gives the possibility of producing a hydrogen stream and an oxygen stream by electrolysis of the water. The water feeding the electrolysis unit 22 from a water line 72 stems from a water source outside the process. In order to limit total water consumption of the process, a portion of this water supply may also stem from the gas conditioning unit 12, and/or from the first separation unit 18. The hydrogen leaves the electrolysis unit via line 56. The oxygen leaves the electrolysis unit via line 74, and feeds the gasification unit or the reforming unit. The electrolysis unit 22 is also supplied with electricity via the electric line 76. The electric line 76 is typically connected to an electric distribution network, the network not being dedicated to the electrolysis unit 22 and serving other consumers outside the unit for producing methanol or hydrocarbons.

In the air separation unit 24, oxygen from air is separated from the other gases, such as nitrogen. In certain exemplary embodiments, the air separation unit 24 is not required, the electrolysis unit 22 providing sufficient oxygen for feeding the production unit 10.

The different units of the facility are controlled by a programmed control unit 78 for applying the production method which will be described below.

The production facility of FIG. 2 will now be described. Only the points through which this facility differs from that of FIG. 1 will be detailed below. Identical elements all providing the same function in both facilities will be designated with the same references.

In this facility, the reforming unit 30 is a steam reforming (SR, Steam Reforming) unit. The reforming unit 30 comprises a combustion sub-unit 80 and a reforming sub-unit 82. The intermediate gas stream after passing into the unit for adjusting the steam level 40, feeds the sub-unit 80. In the sub-unit 80, this gas is indirectly heated, by heat exchange with a combustion gas. The heated-up intermediate gas stream leaves the combustion sub-unit 80 through line 84 and is directed to the reforming sub-unit 82. The combustion gas, in the example of FIG. 2, stems from the second separation unit 20. It typically includes CO, CO₂, CH₄ and H₂ and light hydrocarbons. It is directed from unit 20 as far as an addition unit 88, via line 86. In the addition line 88, an oxidizer, for example air or oxygen is added to the combustion gas, via line 90. The combustion gas is then directed via line 92 of unit 88 as far as the combustion sub-unit, in which it burns while releasing heat. The burnt gases leave the sub-unit 80 through line 94 and form the purge. They are discharged into the atmosphere, optionally after purification.

In the reforming sub-unit 82, the methane of the heated-up intermediate gas stream is broken down and converted into CO, CO₂ and H₂ by reforming reactions.

The first stream of synthesis gas, leaving the reforming sub-unit 82, is directed towards the gas conditioning unit through line 32.

In the production method of the invention, modulation of the electric power demand of the electrolysis unit 22 is provided, in order to adapt to the electric power available on the network. Firstly, this gives the possibility of decreasing the electric power of the electrolysis unit during peak periods, i.e. when the other electric consumers connected to the network demand significant power. Indeed, the consumed electric power varies during a same day, with for example a peak at the end of the day. On the other hand, electric consumption during the night is reduced.

Electric consumption also varies during the year, this consumption being greater during winter months because of the large number of consumers having electric heating means, and is more reduced in summer at least in countries where air conditioning is not in widespread use.

Modulating the electric power of the electrolysis unit is also particularly useful in the case when the electricity distribution network is supplied with electric sources of variable power, such as wind turbine or solar sources. The electric power provided by such sources actually varies depending on the weather conditions.

Thus, in the production method of the invention, the electric power consumed for producing the hydrogen stream is more significant at certain so-called off-peak periods and less significant at other so-called peak periods.

Thus, the electrolysis unit 22 produces a smaller hydrogen stream in a peak period and a larger one in an off-peak period.

In order to compensate for this lack of hydrogen and to therefore obtain the proper H₂/CO ratio at the inlet of the unit 16 (typically of the order of 2.1 for a Fischer-Tropsch application), the control unit 78 is programmed so as to apply the following strategy.

The operating conditions of the reforming unit 30 may be modified in order to produce a more or less significant amount of hydrogen. Typically, the H₂/CO molar ratio obtained at the outlet of the unit 30 may vary between 0.6 and 3. Indeed, the operation of this unit is determined by the temperature, the reaction pressure and the composition of the incoming streams in the reforming unit 30, notably the H₂O/C and O₂/C ratio (respectively the total molar flow rate of water entering the unit 30 over the total molar flow rate of carbon atoms contained in the organic compounds entering the unit 30, and the total oxygen molar flow rate entering the unit 30 over the total molar flow rate of carbon atoms contained in the organic compounds entering the unit 30).

By [symbol] total molar flow rate of a species entering the reforming unit 30 [symbol] is meant the sum of the molar flow rates of the species in all the streams entering the unit 30. In the example of FIG. 1, the total molar flow rate of carbon atoms contained in the organic compounds corresponds to the sum of the molar flow rates of carbon atoms in the different organic compounds (hydrocarbons) in the intermediate stream and in the first recycled gas stream. The total molar flow rate of water H₂O corresponds to the sum of the water molar flow rates in the intermediate gas stream 44, 49 and in the first recycled gas stream (48). The total molar flow rate of oxygen essentially corresponds to the oxygen flow rate brought through line 46, since the molar flow rate of oxygen in the other streams entering the reforming unit 30 is practically zero.

In practice, when methane CH₄ is in majority in the organic compounds entering the reforming unit 30, it is possible to approximate the ratios above for H₂O/CH₄ and O₂/CH₄ ratios. H₂O and O₂ have the same meaning as above. CH₄ corresponds to the total molar flow rate of methane in all the streams entering the reforming unit 30.

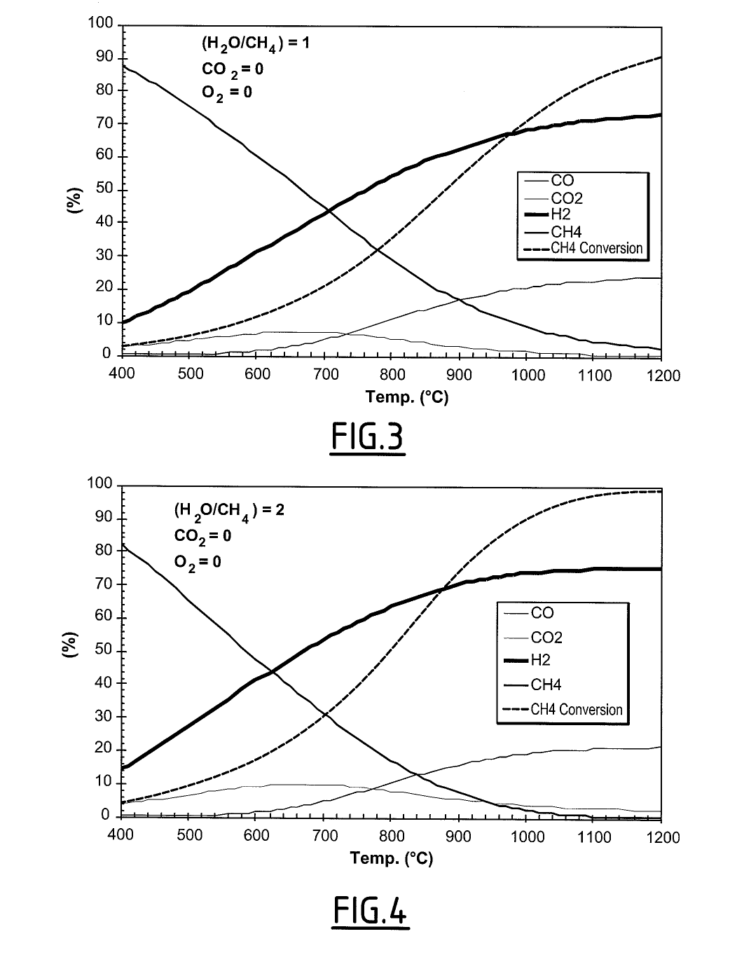

FIGS. 3 to 10 show calculations obtained by simulation in order to illustrate in a simplified way the time-dependent change of the different molar fractions of the gases produced in an ATR or SR type unit. The relevant reactor is isothermal and the operating pressure 30 bars. The thereby obtained concentrations are at thermodynamic equilibrium (Gibbs reactor). Further, it was considered that the reactor was perfectly heated to the desired temperature.

FIGS. 3 to 6 show the time-dependent change in the molar fractions on a dry basis of CH₄, H₂, CO and CO₂ at equilibrium, and the methane conversion level at the outlet of the reforming unit of FIG. 1, versus the reaction temperature. The temperature is in abscissas, the molar fractions and the methane conversion level are in ordinates. The temperature is expressed in degrees C. and the other parameters in %.

For a reforming unit of the ATR type which comprises two steps, integrated in a single reactor:
a partial combustion or oxidation step, in which oxygen is injected in order to increase the temperature of the inflowing gases up to the desired reaction conditions, under the effect of exothermic reactions,
a second endothermic step, during which the reforming reactions take place in a fixed catalytic bed.

The temperatures in the catalytic bed typically vary between 800 and 1,000° C., under a pressure from 20 to 100 bars. In the upper portion of the catalytic bed, the temperatures may attain 1,100 to 1,400° C. The catalyst is for examples based on nickel on a spinel support. The temperature in the combustion step is of the order of 2,000° C.

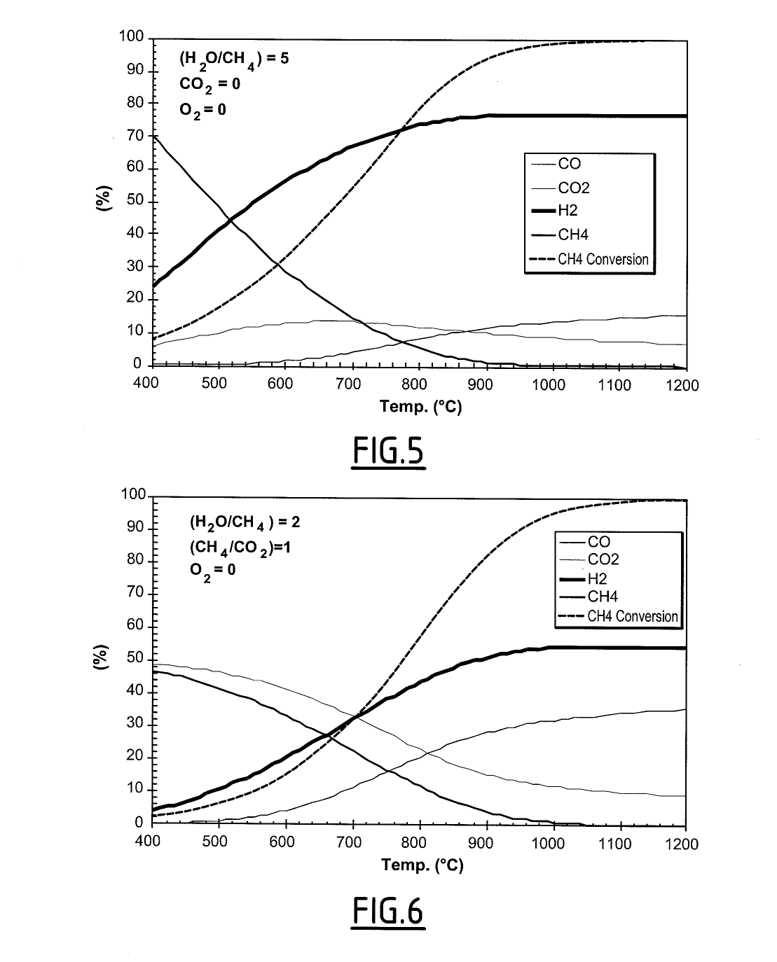

In FIGS. 3, 4 and 5, the stream of inflowing gas does not include any CO₂. On the other hand, the ratio of total H₂O over total CH₄ at the inlet of the reactor is 1 in FIG. 3, 2 in FIG. 4, 5 in FIG. 5. In FIG. 6, the ratio of total H₂O over total CH₄ in the inflowing gases is 2, and the ratio of total H₂O over total CH₄ in the inflowing gases is 1.

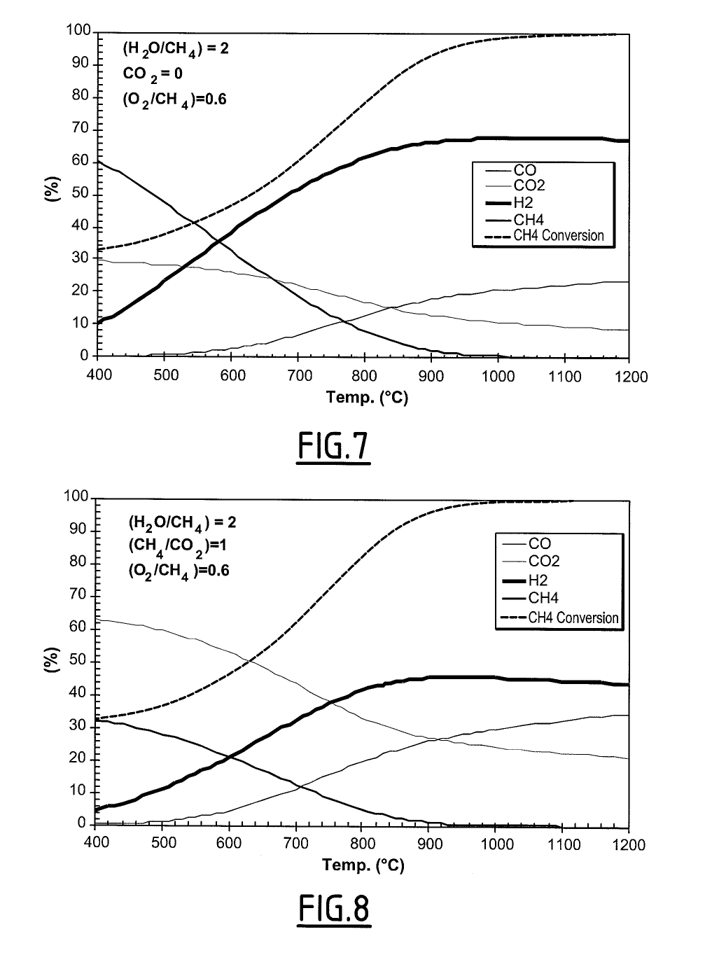

FIGS. 7 and 8 give the same quantities depending on the temperature as FIGS. 3 to 6, for a steam reforming unit such as the one illustrated in FIG. 2. In FIGS. 7 and 8, the ratio of total H₂O over total CH₄ at the inlet of the reforming unit is equal to 2. Also, the ratio of total O₂ over total CH₄ at the inlet of the reforming unit has the value 0.6 in both cases. On the other hand for FIG. 7, there is no CO₂ in the gases entering the reforming unit. For FIG. 8, the ratio of total CH₄ over total CO₂ at the inlet of the reforming unit has the value 1.

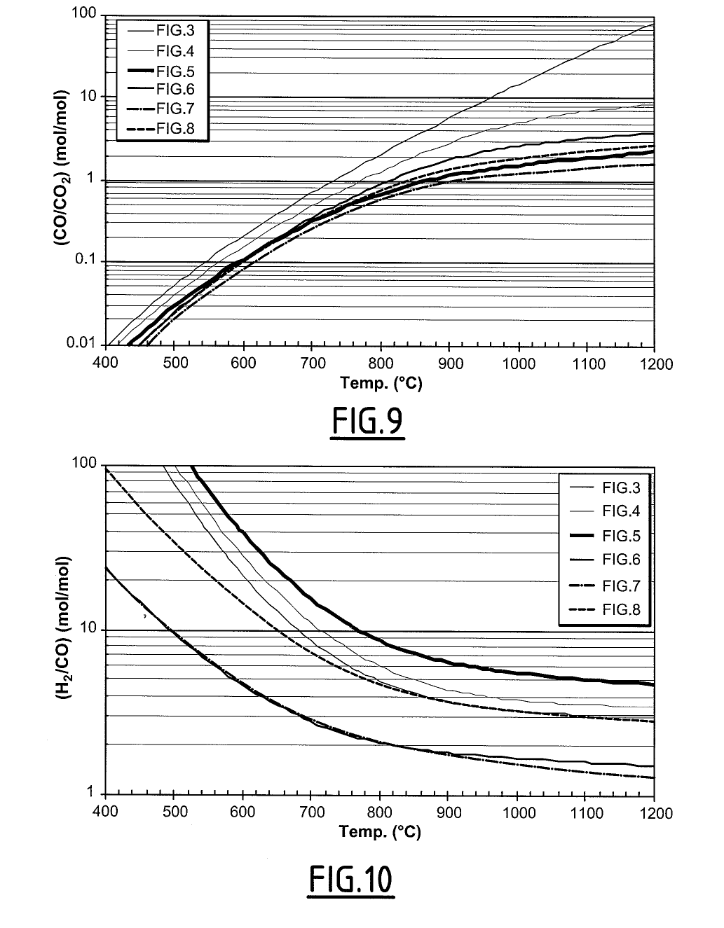

FIGS. 9 and 10 show the time-dependent change in the respective CO/CO₂ and H₂/CO ratios at equilibrium, i.e. at the outlet of the reactor, versus the reaction temperature for the six cases corresponding to FIGS. 3 to 8.

It clearly emerges that:
a) The methane conversion level is very sensitive to the reaction temperature and conversion of the methane is promoted by high temperatures,
b) A large (H₂O/CH₄) ratio promotes conversion of methane and therefore production of synthesis gas (FIGS. 3, 4 and 5),
c) The presence of CO₂ and of O₂ at the inlet of the reactor causes a reduction of the (H₂/CO) ratio as well as their obtained relative amounts (FIGS. 7 and 8),
d) The CO₂ level increases with the increase in the (H₂O/CH₄) ratio at the inlet of the reactor (FIGS. 3, 4 and 5),
e) In FIGS. 9 and 10, it may be seen that the presence of O₂ at the inlet of the reformer causes a significant reduction in the H₂/CO ratio.
f) The H₂/CO ratio decreases with the reaction temperature,
g) In the whole of the figures, it may be seen that there exists a compromise between the conversion of methane and the obtained H₂/CO ratio.

Conversion of the methane is promoted for lower pressures. However, this solution for varying the H₂/CO ratio is not preferential, since a significant decrease in the operating pressure would imply addition of a compression unit downstream in order to obtain a suitable pressure for the unit 16 (typically between 25 and 35 bars for a Fischer-Tropsch unit).

Further, although the reaction temperature is a parameter which highly influences the H₂/CO ratio, this parameter may be varied with caution, since the reaction kinetics, which depends on the activity of the catalyst, and the reaction rates are modified accordingly.

Thus, in order to compensate for a decrease in the consumed electric power by the electrolysis unit 22, and therefore a decrease in the amount of hydrogen produced by this unit 22, the operating temperature of the reforming unit 30 should be reduced and/or the O₂/C ratio should be decreased at the inlet of the reforming unit 30 and/or the H₂O/C ratio should be increased at the inlet of the reforming unit.

In a less preferential alternative, the operating pressure of the reforming unit 30 should be decreased.

In practice, it is not possible to carry out all these modifications simultaneously, or only in restricted ranges. Indeed, a decrease in the temperature affects the reaction kinetics and it is possible that equilibrium conditions at the outlet of the reactor are not attained for too low temperatures. Moreover, the oxygen amount determines the reaction temperature of the reformer and an increase in the H₂O/C ratio implies oxygen demand, since there is a larger proportion of reforming reactions (endothermic reactions). Thus, it is necessary to determine on a case by case basis, depending on the composition of the intermediate gas and on the reforming unit type used, which parameters may be acted upon and to which extent.

FIG. 10 shows that, for the relevant calculation cases in FIGS. 6 to 8, an increase in the ratio of total H₂O over total CH₄ at the inlet of the reforming unit allowed an increase in the H₂O/C ratio at the outlet of the reforming unit. The control unit 78 is programmed in order to vary the total H₂O/C (or H₂O/CH₄) ratio at the inlet of the reforming unit by at least one factor comprised between 1.1 and 5, preferably comprised between 1.5 and 3.

However, in certain other exemplary embodiments, for example with other types of carbonaceous material and other operating conditions of the reforming unit, the total H₂O/total CH₄ ratio at the inlet of the reforming unit should be decreased by the control unit 78 in order to increase the H₂/CO ratio at the outlet of the reforming unit.

Depending on the cases, it will be necessary to increase or decrease the O₂/CH₄ ratio at the inlet of the reforming unit in order to increase the H₂O/CO ratio at the outlet of this same reforming unit.

In the example shown below in Table 1, the only parameter for controlling the H2/CO ratio at the outlet of the unit 30 is the H₂O/C ratio. The value of the O₂/C ratio is adjusted so as to have constant reforming temperature.

Table 1 shows the performances of the methods of FIG. 1 and of FIG. 2 during a peak period and during an off-peak period when the unit 16 is a Fischer-Tropsch reactor. In the four calculation cases, the H₂/CO ratio at the inlet of the unit 16 is 2.1. In the off-peak period, the consumed electric power by the electrolysis unit is significant (of the order of 150 MW). In a peak period, the electric power consumed by the electrolysis unit is reduced to about 80 MW, but the H₂O/CH₄ ratio at the inlet of the reforming unit is increased. This ratio has the value 1.15 in an off-peak period and the value 5 in a peak period. A decrease in the net power required by the electrolyzer is seen to be of the order of 45%. However, inevitably the FT products decrease by the order of 17%. Indeed, the carbon yield decreases because a portion of the CO is converted into CO₂ and H₂ in order to make up for the lack of electrolytic hydrogen. Also, the discharged amount of CO₂ of the system increases. This CO₂ may be stored so as to be used in another type of recycling operation or for sequestration.

The composition of the intermediate gas stemming from the gasification unit is indicated in Table 2, as well as the mass flow rate of the intermediate gas stream, the temperature and the pressure at the outlet of the gasification unit.

The table shows that the H₂/CO ratio at the outlet of the reforming unit may vary from 0.7 in off-peak periods to 1.2 in peak periods, for the relevant calculation case. The product flow rate at the outlet of the Fischer-Tropsch unit 16 is of the order of 3.4 kg/s in off-peak periods and 2.8 in peak periods.

The method of the invention and the corresponding production facility have multiple examples.

It gives the possibility of adapting the power required by the electrolyzer to the availabilities of the electric distribution network, which contributes to stabilization of the operation of the distribution network at least locally. Moreover, this allows a reduction in the electric consumption of the electrolyzer at the moment when the prices of electricity are the highest, in order to increase electric consumption at the moment when the electricity costs are lower. The yield of the facility is thereby increased. The possibility of modulating the consumed electric power by the electrolysis unit allows the latter to operate with electricity not stemming from a local electric distribution network but directly from a renewable electric source, for example wind turbines.

It is possible to adapt the operation of a facility without resorting to massive storage of hydrogen or of electric power.

The electric power consumed by the electrolyzer may be strongly reduced, the amount of produced hydrocarbon being moderately reduced. The amount of hydrocarbon produced per consumed MW is clearly greater in a peak period than in an off-peak period, as shown by Table 1. In the examples of this table, a reduction by more than 40% in the electric power consumed by the electrolysis unit causes a reduction by at least 20% of the produced amount of hydrocarbons.

As compared with these results, if the flexibility of the method towards the supply flow rate for the Fischer-Tropsch reactor is also taken into account, it is possible to reach a reduction by more than 50% of the consumed electric power per electrolysis unit, for a reduction by less than 30% of the amount of produced hydrocarbons.

The method and the production facility may have multiple alternatives.

The production unit 16 may not operate according to the Fischer-Tropsch process but be a unit for producing methanol. Methanol may be the final product or be subject to a post-treatment in order to be converted into a hydrocarbon, for example according to the MTG (Methanol to Gasoline) process.

The air separation unit 24 is not required in every case, the electrolysis unit being in certain cases sufficient for providing the required amount of oxygen to the gasification unit and to the reforming unit.

TABLE 1
Comparison of the performances of the FT method with an ATR and SR unit

| | FIG. 2 (SR) | FIG. 1 (ATR) | FIG. 2 (SR) | |
|---|---|---|---|---|
| | Off-peak | Off-peak | Peak | FIG. 1 (ATR) |
| | period | period | period | Peak period |
| Study variables | | | | |
| (H₂/CO) (mol) Line 60 | 2.1 | 2.1 | 2.1 | 2.1 |
| (H₂O/CH₄) (mol) | 1.15 | 1.15 | 5 | 5 |
| Reformer inlet Line 44 | | | | |
| ATR or SR (Unit 30) | | | | |
| (H₂/CO) (mol) reformer | 0.69 | 0.73 | 1.20 | 1.19 |
| outlet Line 32 | | | | |
| O₂ reformer supply (kg/s) | — | 3.392 | — | 2.478 |
| Line 46 | | | | |
| Combustion | | | | |
| O₂ reformer (kg/s) Line 90 | 3.697 | — | 2.522 | — |
| Electrolyzer (Unit 22) | | | | |
| H₂ (kg/s) Line 56 | 0.880 | 0.846 | 0.476 | 0.4682 |
| O₂ (kg/s) Line 74 | 6.987 | 6.714 | 3.778 | 3.716 |
| MW consumed Line 76 | 152 | 146 | 82 | 81 |
| FT process (Unit 16) | | | | |
| Conversion (%) | 70 | 70 | 70 | 70 |
| Synthesis gas (CO + H₂) | 9.11 | 9.00 | 7.91 | 7.73 |
| supply (kg/s) Line 50 | | | | |
| FT process supply (kg/s) | 12.43 | 12.37 | 11.39 | 11.29 |
| Line 50 | | | | |
| FT reactor supply (kg/s) | 32.33 | 32.40 | 30.52 | 30.63 |
| Line 60 | | | | |
| Products (kg/s) Line 64 | 3.402 | 3.354 | 2.847 | 2.781 |
| Purge ratio (%) | 19 | 5 | 17 | 5 |
| Gas purge (kg/s) Line | 4.517 | 1.199 | 3.693 | 1.180 |
| 70/94 | | | | |
| Discharged CO₂ (kg/s) | 1.945 | 6.859 | 4.757 | 8.679 |
| Line 52 | | | | |
| (kg of products)/(MWh of | 80.5 | 82.6 | 125.0 | 123.6 |
| electrolyzer) | | | | |

*The ATR and SR units operate at 950° C. and 28.5 bars.

TABLE 2
Dry basis composition of the intermediate
gas at the outlet of the gasification unit

| | | |
|---|---|---|
| CO | 19.2% | mol |
| CO₂ | 45.0% | mol |
| H₂ | 19.0% | mol |
| CH₄ | 13.2% | mol |
| Other organic compounds | 3.5% | mol |
| H₂/CO | 1.00 | mol/mol |
| Pressure | 29 | bar |
| Temperature | 950° | C. |
| Mass flow rate | 20.4 | kg/s |
| Ethane | 0.08% | v/v |
| Ethylene | 1.53% | v/v |
| Methane | 8.17% | v/v |
| CO | 11.86% | v/v |
| CO₂ | 27.92% | v/v |
| H₂ | 11.79% | v/v |
| H₂O | 37.69% | v/v |
| N₂ | 0.06% | v/v |
| Ar | 0.06% | v/v |
| Ammonia | 0.29% | v/v |
| H₂S | 0.01% | v/v |
| BTX - Benzene | 0.26% | v/v |
| Tars-Naphthalene | 0.27% | v/v |
| H₂/CO | 1.00 | mol/mol |
| H₂O/CH₄ | 4.6 | mol/mol |

What is claimed is:

1-10. (canceled)

11: A method for producing at least one of methanol and hydrocarbons from at least one carbonaceous material, the method comprising:
producing a stream of synthesis gas from the carbonaceous material, according to a process comprising at least one operation for reforming an intermediate gas stream from the carbonaceous material, the stream of synthesis gas comprising at least hydrogen and carbon monoxide, the synthesis gas having a first hydrogen/carbon monoxide molar ratio under first operating conditions for performing the reforming;
producing a hydrogen stream from a hydrogenated raw material and from a first consumed electric power, the hydrogen stream having a first molar flow rate for the first consumed electric power;
producing at least one of methanol and hydrocarbons from the synthesis gas and from the hydrogen stream; and
decreasing the consumed electric power for producing the hydrogen stream, down to a second consumed electric power below the first electric power, the hydrogen stream having a second molar flow rate below the first molar flow rate for the second consumed electric power, and setting second operating conditions different from the first operating conditions for the reforming operation in order to compensate for the decrease of the molar flow rate of the hydrogen stream, the synthesis gas having a second hydrogen/carbon monoxide molar ratio greater than the first hydrogen/carbon monoxide molar ratio under the second operating conditions.

12: The method as recited in claim 11 wherein, under the first operating conditions, the reforming operation is carried out at a first temperature, under the second operating conditions, the reforming operation being carried out at a second temperature below the first temperature.

13: The method as recited in claim 11 wherein, under the first operating conditions, the reforming operation is carried out at a first pressure, under the second operating conditions, the reforming operation is carried out at a second pressure below the first pressure.

14: The method as recited in claim 1 wherein the reforming operation is carried out in a reformer receiving at an inlet of the reformer a plurality of inflows containing organic molecules, including the intermediate gas stream, the reformer receiving from the different inflows, all in all, a total molar flow rate of carbon atoms contained in the organic molecules and a total water H₂O molar ratio, the reforming operation passing from the first operating conditions to the second operating conditions by modifying a ratio of the total water H₂O molar flow rate over the total molar flow rate of carbon atoms contained in the organic molecules.

15: The method as recited claim 14 wherein the ratio of the total water H₂O molar flow rate over the total molar flow rate of carbon atoms contained in the organic molecules is equal to a first value under the first operating conditions, and is equal to a second value greater than the first value under the second operating conditions.

16: The method as recited claim 15 wherein the second value is comprised between 1.1 and 5 times the first value.

17: The method as recited in claim 16 wherein the second value is comprised between 1.5 and 3 times the first value.

18: The method as recited in claim 11 wherein the reforming operation is carried out in a reformer receiving at an inlet of the reformer a plurality of inflows, including the intermediate gas stream, the reformer receiving from the different inflows, all in all, a total molar flow rate of carbon atoms contained in the organic molecules and a total oxygen O₂ molar flow rate, the reforming operation passing from the first operating conditions to the second operating conditions by modifying a ratio of the total molar flow rate of oxygen over the total molar flow rate of carbon atoms contained in the organic molecules.

19: The method as recited in claim 11 wherein the hydrogen stream is produced from electricity provided by an electric distribution network serving other consumers.

20: The method as recited in claim 11 wherein that the intermediate gas stream is obtained by gasification of the carbonaceous material.

21: A facility for producing at least one of methanol and hydrocarbons from at least one carbonaceous material, the facility comprising:
a unit for producing synthesis gas from the carbonaceous material, comprising at least one unit for reforming an intermediate gas stream from the carbonaceous mater, the stream from synthesis gas comprising at least hydrogen and carbon monoxide, the synthesis gas having a first hydrogen/carbon monoxide molar ratio under the first operating conditions for the reforming unit;
a unit for producing a hydrogen stream from a hydrogenated raw material and from a first consumed electric power, the hydrogen stream having a first molar flow rate for said first consumed electric power;
a unit for producing at least one of methanol and hydrocarbons from the synthesis gas and from the hydrogen stream;
a programmed control unit for decreasing the consumed electric power for producing the hydrogen stream, down to a second electric power below the first electric power, the hydrogen stream having a second molar flow rate below the first molar flow rate for the second consumed electric power, and for setting the reforming unit under second operating conditions different from the first for compensating for the decrease in the molar flow rate of the hydrogen stream, the synthesis gas having a second hydrogen/carbon monoxide molar ratio greater than the first under the second operating conditions.

22: A facility for producing at least one of methanol and hydrocarbons from at least one carbonaceous material, the facility comprising:
a synthesis gas producer producing synthesis gas from the carbonaceous material, comprising at least one reformer for reforming an intermediate gas stream from the carbonaceous matter, the stream from synthesis gas comprising at least hydrogen and carbon monoxide, the synthesis gas having a first hydrogen/carbon monoxide molar ratio under the first operating conditions for the reformer;
a hydrogen producer producing a hydrogen stream from a hydrogenated raw material and from a first consumed electric power, the hydrogen stream having a first molar flow rate for said first consumed electric power;
an at least one of methanol and hydrocarbons producer producing at least one of methanol and hydrocarbons from the synthesis gas and from the hydrogen stream;
a programmed controller decreasing the consumed electric power for producing the hydrogen stream, down to a second electric power below the first electric power, the hydrogen stream having a second molar flow rate below the first molar flow rate for the second consumed electric power, and for setting the reformer under second operating conditions different from the first for compensating for the decrease in the molar flow rate of the hydrogen stream, the synthesis gas having a second hydrogen/carbon monoxide molar ratio greater than the first under the second operating conditions.
